(19) United States
(12) Patent Application Publication
(10) Pub. No.: US 2018/0232589 A1
(43) Pub. Date: Aug. 16, 2018

(54) DEVICE FOR MEASURING BIOMETRIC INFORMATION AND INTERNET OF THINGS SYSTEM INCLUDING THE SAME

(71) Applicant: Samsung Electronics Co., Ltd., Suwon-si (KR)

(72) Inventors: Jong Hyuk Woo, Yongin-si (KR); Seung Jae Lee, Hwaseong-si (KR); Yong In Park, Seoul (KR); Han Shin Shin, Yongin-si (KR); Seoung Jae Yoo, Seongnam-si (KR); Kwan Hee Lee, Seoul (KR)

(21) Appl. No.: 15/801,664

(22) Filed: Nov. 2, 2017

(30) Foreign Application Priority Data
Feb. 16, 2017 (KR) 10-2017-0021002
May 24, 2017 (KR) 10-2017-0064029

(51) Int. Cl. G06K 9/00; H04L 29/06; A61B 5/053; A61B 5/0402; A61B 5/0488; A61B 5/1171; A61B 5/1172

(52) U.S. Cl. CPC G06K 9/00885; H04L 63/0861; A61B 5/0531; G06K 9/6202; A61B 5/0488; A61B 5/1171; A61B 5/1172; A61B 5/0402

(57) ABSTRACT

A device for measuring biometric information includes an impedance measurement circuit, a storage circuit, and a control circuit. The impedance measurement circuit receives an electrical signal from a plurality of electrodes when the plurality of electrodes are in contact with a body of a user and measures a bioelectrical impedance using the electrical signal. The storage circuit includes a security area configured to store the bioelectrical impedance, body information of the user, and biometric information of the user obtained from the bioelectrical impedance and the body information. The control circuit includes an authentication means configured to determine access authority to data stored in the security area using an authentication procedure, obtains the biometric information from the bioelectrical impedance and the body information, and manages the security area through the authentication procedure provided by the authentication means.

### CROSS-REFERENCE TO RELATED APPLICATION

This application claims priority under 35 U.S.C. § 119 to Korean Patent Application No. 10-2017-0021002, filed on Feb. 16, 2017, and Korean Patent Application No. 10-2017-0064029, filed on May 24, 2017 in the Korean Intellectual Property Office, the disclosures of which are incorporated by reference herein in their entireties.

### TECHNICAL FIELD

Exemplary embodiments of the present inventive concept relate to a device for measuring biometric information and an Internet of Things (IoT) system including the same.

### DISCUSSION OF RELATED ART

As biosensors capable of obtaining users' biometric information have become widespread, various applications using such biometric information have been developed. For example, electronic devices having biosensors may directly provide biometric information, obtained by the biosensors, to users. However, biometric information may include very sensitive personal information related to users, and thus, when providing various applications using biometric information to users, proper security solutions should be provided with these applications.

### SUMMARY

According to an exemplary embodiment of the present inventive concept, a device for measuring biometric information may include an impedance measurement circuit, a storage circuit, and a control circuit. The impedance measurement circuit may be configured to receive an electrical signal from a plurality of electrodes when the plurality of electrodes are in contact with a body of a user and to measure a bioelectrical impedance using the electrical signal. The storage circuit may include a security area to store the bioelectrical impedance, body information of the user, and biometric information of the user obtained from the bioelectrical impedance and the body information. The control circuit may include an authentication means to determine access authority to data stored in the security area using an authentication procedure, and may be configured to obtain the biometric information from the bioelectrical impedance and the body information and to manage the security area through the authentication procedure provided by the authentication means.

According to an exemplary embodiment of the present inventive concept, a device for measuring biometric information may include an electrode circuit including a plurality of electrodes configured to be in contact with a body of a user, an electrocardiogram (ECG) sensor configured to measure an ECG signal using an electrical signal detected by the electrode circuit, and a control circuit including an authentication means. The authentication means may be configured to provide a first authentication procedure and a second authentication procedure, and to authenticate the user that has passed the first authentication procedure and the second authentication procedure as an authenticated user. The first authentication procedure may authenticate the user with a first authentication method using the ECG signal. The second authentication procedure may authenticate the user with a second authentication method that does not use the ECG signal.

According to an exemplary embodiment of the present inventive concept, an Internet of Things (IoT) system may include a plurality of electronic devices configured to communicate with one another through an IoT network, and at least one biometric information measurement device configured to communicate with at least a portion of the plurality of electronic devices through the IoT network, and to obtain biometric information of a user through contact with a body of the user. The at least one biometric information measurement device may include an authentication means configured to confirm whether the user is authenticated, and may share the biometric information with the at least a portion of the plurality of electronic devices through an authentication procedure provided by the authentication means. The plurality of electronic devices may provide services to the user using the biometric information.

### BRIEF DESCRIPTION OF DRAWINGS

The above and other features of the present inventive concept will be more clearly understood by describing in detail exemplary embodiments thereof with reference to the accompanying drawings.

FIG. 1 is a schematic diagram of an Internet of Things (IoT) system according to an exemplary embodiment of the present inventive concept.

FIGS. 2 through 5 are diagrams illustrating operations of an IoT system according to exemplary embodiments of the present inventive concept.

FIG. 6 is a flowchart illustrating operations of a device for measuring biometric information included in an IoT system according to an exemplary embodiment of the present inventive concept.

FIG. 7 is a diagram illustrating operations of devices for measuring biometric information according to an exemplary embodiment of the present inventive concept.

FIGS. 8 and 9 are schematic block diagrams illustrating devices for measuring biometric information according to exemplary embodiments of the present inventive concept.

FIGS. 10 and 11 are diagrams illustrating operations of devices for measuring biometric information according to exemplary embodiments of the present inventive concept.

FIGS. 12 and 13 are flowcharts of user authentication methods of a device for measuring biometric information according to exemplary embodiments of the present inventive concept. and

FIG. 14 is a graph of an electrocardiogram (ECG) signal used in the user authentication method of FIG. 13 according to an exemplary embodiment of the present inventive concept.

### DETAILED DESCRIPTION OF THE EMBODIMENTS

Exemplary embodiments of the present inventive concept may provide a security solution which may safely manage biometric information obtained by a device for measuring the biometric information. Exemplary embodiments of the present inventive concept may also provide various applications using the biometric information and the security solution in an Internet of Things (IoT) environment.

Exemplary embodiments of the present inventive concept will be described hereinafter with reference to the accompanying drawings. Like reference numerals may refer to like elements throughout this application.

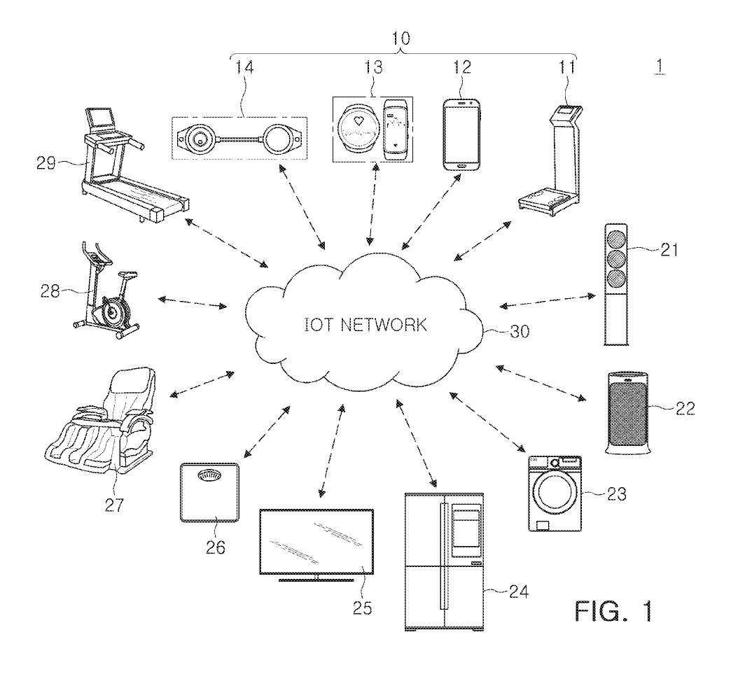

FIG. 1 is a schematic diagram of an Internet of Things (IoT) system, according to an exemplary embodiment of the present inventive concept.

Referring to FIG. 1, an IoT system 1, according to an exemplary embodiment of the present inventive concept, may include biometric information measurement devices 10 able to obtain a user's biometric information, and a plurality of electronic devices 21 to 29. The biometric information measurement devices 10 may obtain the biometric information when in contact with the user's body. To obtain the biometric information, the biometric information measurement devices 10 may include at least one electrode in contact with the user's body.

The electronic devices 21 to 29 may be connected to one another to communicate with one another through an IoT network 30. The electronic devices 21 to 29 may be connected to the biometric information measurement devices 10 through the IoT network 30 to communicate with the biometric information measurement devices 10. The electronic devices 21 to 29 may not include a function of obtaining the biometric information from the user, unlike the biometric information measurement devices 10. For communications over the IoT network 30, each of the biometric information measurement devices 10 and the electronic devices 21 to 29 may include a communications module supporting a wired/wireless communications function, and a storage unit storing information required to build an IoT environment and to provide applications.

The biometric information measurement devices 10 may include a fixed measurement device 11, a smartphone 12 as a mobile device, and wearable devices 13 and 14. The wearable devices 13 and 14 may include a smartwatch 13 or a patch 14.

The fixed measurement device 11 may include a plurality of electrodes in contact with soles or palms of the user. For example, the fixed measurement device 11 may obtain the biometric information when the electrodes come into contact with the soles of the feet when the user stands on a footboard of the fixed measurement device 11 or the palms of the hands when the user places his or her hands on the fixed measurement device 11.

The smartphone 12 may include electrodes provided for the user to bring his or her finger or the like into contact therewith, and the user may hold the smartphone 12 such that the user's body may come into contact with the electrodes disposed in a plurality of positions on the smartphone 12. Thus, the smartphone 12 may obtain the biometric information from the user.

The smartwatch 13 or the patch 14 may include at least one electrode to be in contact with the user's body continuously, to acquire the biometric information.

The biometric information measurement devices 10 may supply a fine level of current to the user's body through the electrodes in contact therewith and measure a resulting level of voltage so as to measure bioelectrical impedance of the user. The biometric information measurement devices 10 may obtain the biometric information using the bioelectrical impedance and the user's body information. In an exemplary embodiment of the present inventive concept, the body information may include information about the user's age, sex, height, and/or weight. The biometric information, obtained using the bioelectrical impedance and the user's body information, may include at least one of the user's body fat mass, body water content, muscle mass, heart rate, stress information, skin temperature, or skin dryness.

The biometric information, obtained by the biometric information measurement devices 10, may be shared with the electronic devices 21 to 29 through the IoT network 30. The electronic devices 21 to 29 may receive the biometric information through the IoT network 30, and may provide various applications to the user, based on the received biometric information.

The electronic devices 21 to 29 may include an air conditioner 21, a humidifier 22, a washing machine 23, a refrigerator 24, a television 25, a body weight scale 26, a massage chair 27, and exercise machines 28 and 29, which will be described further below.

In an exemplary embodiment of the present inventive concept, when it is determined that a stress level of the user is high, the IoT system 1 may recommend that the user use the massage chair 27 via notification by the smartphone 12 or the smartwatch 13. When the user sits on the massage chair 27, the massage chair 27 may determine the intensity of massage according to the stress level received from at least one of the biometric information measurement devices 10.

Further, in an exemplary embodiment of the present inventive concept, the IoT system 1 may compare a target weight, target muscle mass, or target body fat mass, previously entered by the user, with the biometric information obtained by the biometric information measurement devices 10, and may provide exercise guidance information to the user. When the target weight, target muscle mass, or target body fat mass, previously set by the user, falls short of the biometric information obtained by the biometric information measurement devices 10, the IoT system 1 may recommend that the user use the exercise machines 28 or 29 via notification by the smartphone 12 or the smartwatch 13. In an exemplary embodiment of the present inventive concept, the IoT system 1 may also guide the user through a proper exercise, an exercise time, or exercise intensity via notification by the smartphone 12 or the smartwatch 13.

The previously set target weight, target muscle mass, or target body fat mass may also be used in a nutrition guidance service using a display of the refrigerator 24. When the target weight, the target muscle mass, or target body fat mass, previously set by the user, falls short of the biometric information obtained by the biometric information measurement devices 10, the IoT system 1 may inform the user about a proper diet through the display of the refrigerator 24. The refrigerator 24 may identify food ingredients therein using an image sensor or a near field communication (NFC) tag function, and may provide the user with recipes for meals that may be made using the food ingredients.

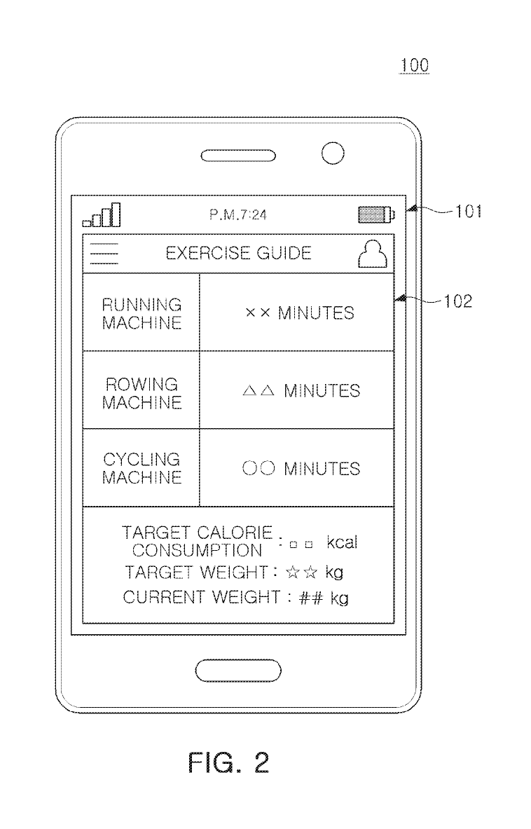

FIGS. 2 through 5 are diagrams illustrating operations of an IoT system according to exemplary embodiments of the present inventive concept.

Referring to FIG. 2, a display 101 of a smartphone 100 connected to the IoT system may be used to display an exercise guidance service 102. The exercise guidance service 102 may include information about a type of exercise, a recommended exercise time, a target weight, and target calorie consumption determined to be suitable for the user, based on the user biometric information.

The smartphone 100 may receive the biometric information from other electronic devices connected to an IoT network, or may obtain the biometric information directly. The biometric information may include information about body water content, body fat mass, or muscle mass calculated by measuring bioelectrical impedance of the user, with information directly entered by the user, such as the user's height, age, or sex.

For example, when the user executes an exercise guidance application on the smartphone 100, the smartphone 100 may compare a target weight, previously entered by the user, with a current weight of the user, and may calculate a target calorie consumption, based on the difference between the target weight and the current weight. The smartphone 100 may display a type of exercise and a resulting exercise time required to achieve the target calorie consumption as the exercise guidance service 102.

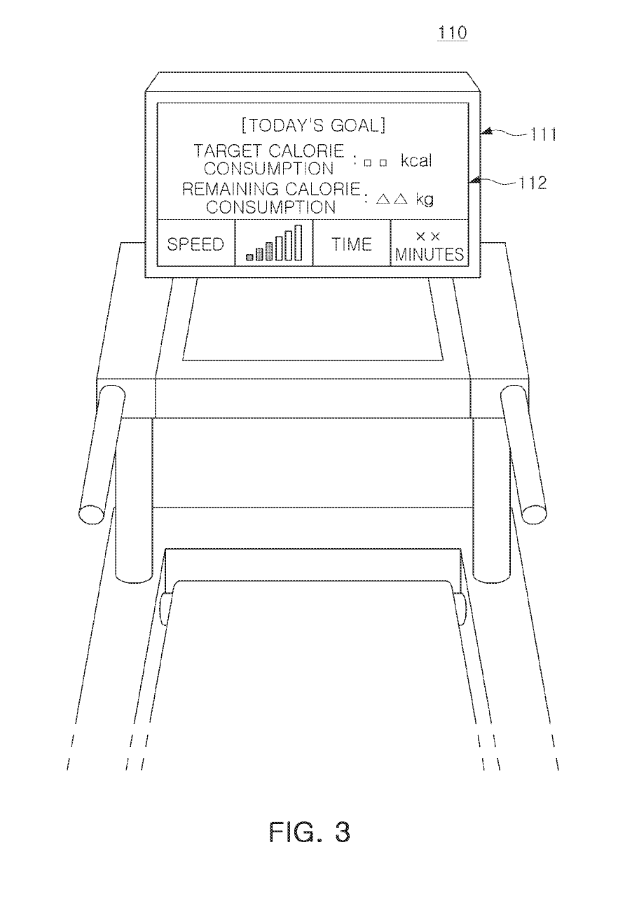

The exercise guidance service 102, displayed on the smartphone 100, may also be similarly displayed on a display 111 of an exercise machine 110 (refer to FIG. 3) used by the user.

Referring to FIG. 3, an exercise guidance service 112 may be displayed on the display 111 of the exercise machine 110. In FIG. 3, the exercise machine 110 may be illustrated as a running machine, but the inventive concept is not limited thereto. For example, the exercise machine 110 may be a cycling machine or a rowing machine. In an exemplary embodiment of the present inventive concept, the exercise guidance service 112 displayed on the display 111 of the exercise machine 110 may indicate target calorie consumption, remaining calorie consumption, or a remaining exercise time.

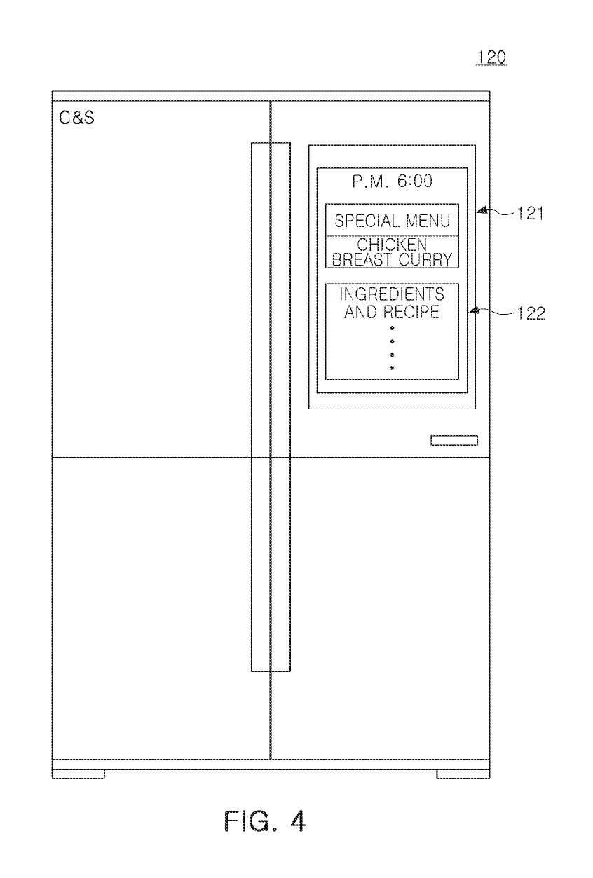

Referring to FIG. 4, an IoT system according to an exemplary embodiment of the present inventive concept may include a refrigerator 120. The refrigerator 120 may include a communications module or the like, providing an IoT function. Further, the refrigerator 120 may include equipment that may identify types or states of food ingredients stored therein.

In an exemplary embodiment of the present inventive concept, the refrigerator 120 may include an image sensor that may capture an image of an internal state thereof, and may identify approximate types or states of food ingredients using the captured image.

In an exemplary embodiment of the present inventive concept, the refrigerator 120 may use an NFC tag to monitor types or states of food stored therein. The user may scan NFC tags attached to containers or packaging of food or food ingredients with an NFC module of the refrigerator 120, and store the food or food ingredients in the refrigerator 120. Thus, the refrigerator 120 may monitor the food or food ingredients stored therein.

The refrigerator 120 may receive the user's biometric information through an IoT network, and may display a diet recommendation service 122 on a display 121 based on the biometric information. The refrigerator 120 may inform the user about recommended meals that may be made using the stored food or food ingredients through the diet recommendation service 122. In an exemplary embodiment of the present inventive concept, when a rate of weight increase or decrease with respect to a target weight previously entered by the user is low, the refrigerator 120 may display a recipe for a meal, containing an amount of calories suitable to allow the user to reach his or her target weight, as the diet recommendation service 122.

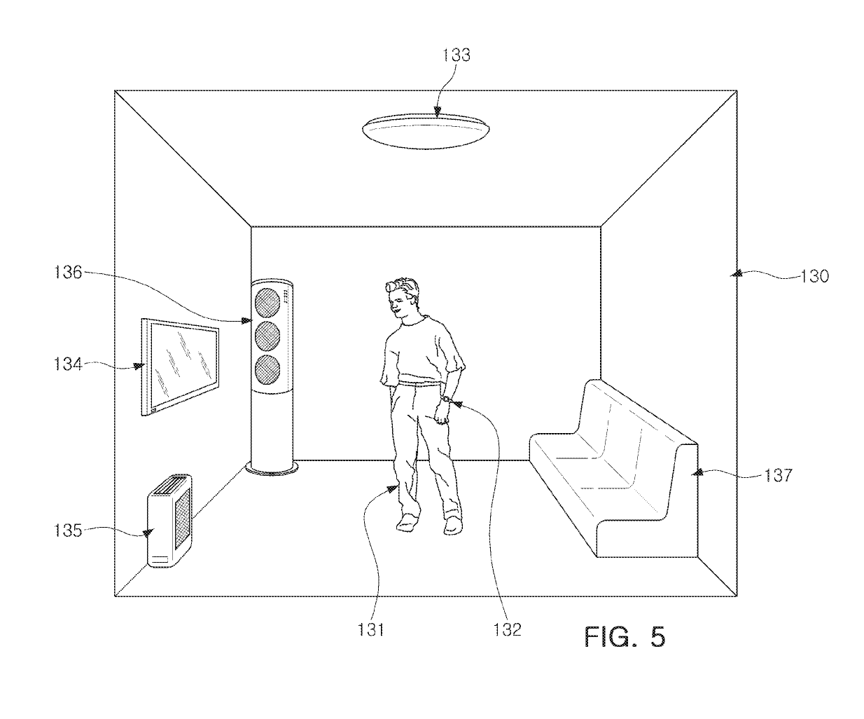

Referring to FIG. 5, an IoT system according to an exemplary embodiment of the present inventive concept may provide a service for managing a predetermined interior space 130. A user 131 may wear a wearable device 132 in contact with the user's body. In FIG. 5, the wearable device 132 may be illustrated as a smartwatch, but may be replaced with various types of devices such as clothing, footwear, or a patch.

The interior space 130, having the IoT system according to an exemplary embodiment of the present inventive concept, may be provided with devices 133 to 137, including a lighting device 133, a television 134, a humidifier 135, an air conditioner 136, and a sofa 137. The wearable device 132 may collect biometric information from the user 131, and the devices 133 to 137 disposed in the interior space 130 may operate based on the biometric information. In an exemplary embodiment of the present inventive concept, a target temperature may be adjusted according to a temperature of the user, and operations of the humidifier 135 may be controlled according to skin dryness of the user. Alternatively or additionally, a recline angle of the sofa 137 may also be adjusted according to a stress index or heart rate of the user.

In an exemplary embodiment of the present inventive concept, the lighting device 133 and the television 134 may operate in connection with a sleep state of the user. The wearable device 132 may check a sleep pattern or the like of the user and, when the user is determined to be falling asleep with the lighting device 133 or the television 134 turned on, the IoT system may stop operations of the lighting device 133 or the television 134. In an exemplary embodiment of the present inventive concept, the operations of the lighting device 133 or the television 134 may also be determined in connection with the stress index of the user.

As described with respect to FIGS. 2 through 5, various apparatuses included in the IoT system according to exemplary embodiments of the present inventive concept may operate based on the user's biometric information. In exemplary embodiments of the present inventive concept, the refrigerator 120 may provide the diet recommendation service 122 to the user, or the exercise machine 110 or the smartphone 100 may provide the exercise guidance service 112/102 to the user, through comparison between the current muscle mass or the body fat mass of the user and a target value previously set by the user. Further, the IoT system may also determine whether the user is in a sleep state and adjust an operating state of a home appliance such as the lighting device 133 or the television 134.

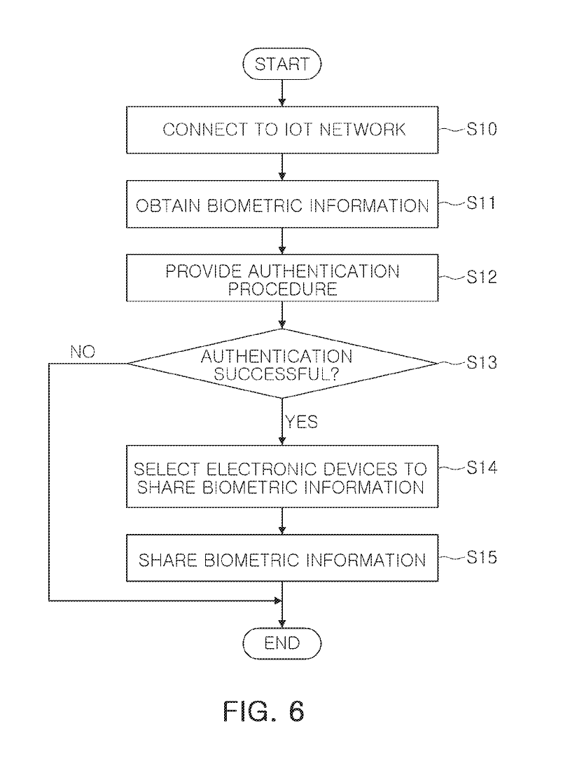

The IoT system according to exemplary embodiments of the present inventive concept may include at least one biometric information measurement device obtaining biometric information through contact with a user's body, and the electronic devices may share the biometric information, obtained by the at least one biometric information measurement device, with one another, through the IoT network. Thus, a proper security policy, for preventing a situation in which the biometric information is shared or leaked without the user's intention, may be applied to the electronic devices included in the IoT system. Hereinafter, a description will be provided with reference to FIG. 6.

FIG. 6 is a flowchart illustrating operations of a device for measuring biometric information included in an IoT system according to an exemplary embodiment of the present inventive concept.

Referring to FIG. 6, the operations of the biometric information measurement device according to an exemplary embodiment of the present inventive concept may start by connecting to an IoT network (S10). The biometric information measurement device may detect an electrical signal when in contact with a user's body, may obtain the user's biometric information from the detected electrical signal, and may include a biosensor for obtaining the biometric information. The biometric information measurement device may be fixed to a certain position on a mobile device, such as a smartphone, to obtain the biometric information, or may be a wearable device such as a smartwatch or a patch.

The biometric information measurement device may include an electrode in contact with the user's body, and may obtain the biometric information using the electrical signal detected through the electrode (S11). In an exemplary embodiment of the present inventive concept, the biometric information measurement device may measure bioelectrical impedance of the user using the electrical signal obtained through contact between the electrode and the user's body. The biometric information measurement device may obtain the biometric information using the bioelectrical impedance and body information entered by the user. The body information, entered by the user, may include the user's age, sex, height, weight, or race, and the biometric information, obtained by the biometric information measurement device, may include information such as body fat mass, body water content, muscle mass, or body mass index (BMI).

The biometric information measurement device may store the biometric information, obtained in S11, in a storage unit. In an exemplary embodiment of the present inventive concept, the biometric information may be stored in a separate security area of the storage unit. The biometric information measurement device may provide a predetermined authentication procedure to the user (S12), may determine whether authentication is successful (S13), and may permit the user to have access to the biometric information stored in the security area only when the user has passed the authentication procedure. The authentication procedure, provided in S12, may employ various authentication methods, such as password input, pattern input, iris recognition, fingerprint recognition, electromyography (EMG) signal detection, or electrocardiogram (ECG) signal detection. In an exemplary embodiment of the present inventive concept, the authentication procedure provided in S12 may also include two or more authentication methods simultaneously or sequentially executed.

As a result of the determination as to whether authentication is successful in S13, when the user succeeds in the authentication, the biometric information measurement device may select electronic devices to share the biometric information with (S14). In an exemplary embodiment of the present inventive concept, the biometric information measurement device may guide the user to directly select the electronic devices to share the biometric information with through the IoT network. When the user selects the electronic devices to share the biometric information with, the biometric information measurement device may share the biometric information with the selected electronic devices (S15).

In contrast, as a result of the determination as to whether authentication is successful in S13, when the user has failed authentication, the biometric information measurement device may not share the biometric information with the electronic devices.

As described with respect to FIG. 6, the biometric information measurement device according to an exemplary embodiment of the present inventive concept may store the biometric information, obtained from the user, in the security area of the storage unit, and may permit the user to have access to the biometric information stored in the security area only when the user has passed the predetermined authentication procedure. Thus, the biometric information, obtained by the biometric information measurement device included in the IoT system, may be managed more safely.

In an exemplary embodiment of the present inventive concept, the electronic devices that have received the biometric information from the biometric information measurement device may also store the biometric information in security areas of storage units thereof. The electronic devices may output the biometric information only when the user has passed self-provided authentication procedures. Alternatively, the electronic devices may output the biometric information only when the user has passed an authentication procedure provided by an electronic device connected through the IoT network or by another electronic device. For example, in an exemplary embodiment of the present inventive concept, all devices included in the IoT system may share or output the biometric information only when the user has passed a predetermined authentication procedure. Thus, security of the biometric information may be increased.

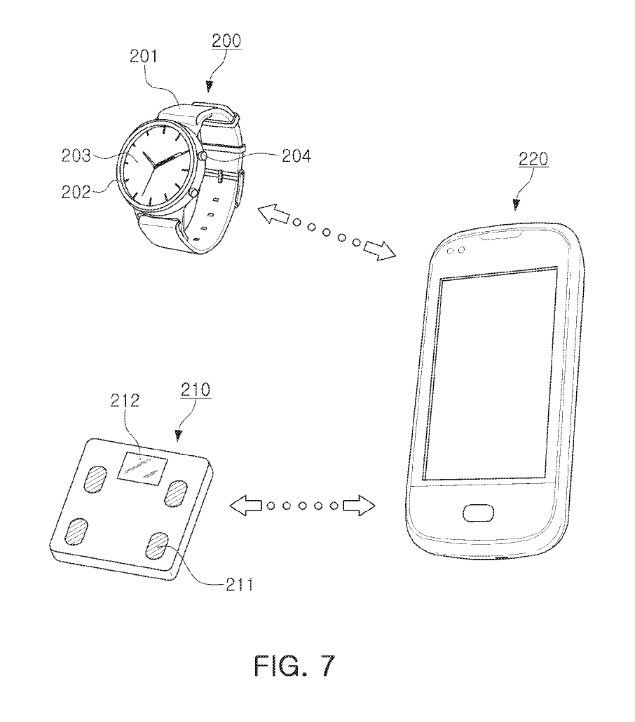

FIG. 7 is a diagram illustrating operations of devices for measuring biometric information according to an exemplary embodiment of the present inventive concept.

Referring to FIG. 7, a biometric information measurement device 200 or 210 may obtain a user's biometric information when in contact with the user's body part. The biometric information measurement device 200 or 210 may be a wearable device, such as a smartwatch 200, which the user may wear, or may be a device, such a body weight scale 210, disposed in a certain location to obtain the biometric information from the user. According to exemplary embodiments of the present inventive concept, the biometric information measurement device 200 or 210 may be provided as various types of devices that may measure biometric information other than the smartwatch 200 and the body weight scale 210.

The smartwatch 200 may include a strap 201 fixing the smartwatch 200 to the user's body and a body portion 202, and the body portion 202 may include a display 203 and a crown 204. The crown 204 may be provided as an input unit for operating various applications, with a touchscreen included in the display 203.

The smartwatch 200 may include a first electrode disposed on an internal surface of the strap 201 or the body portion 202 to constantly be in contact with the user's body, and a second electrode disposed on an external surface of the strap 201 or the body portion 202 such that the user may selectively bring his or her body into contact with the second electrode. According to an exemplary embodiment of the present inventive concept, the second electrode may be built into the crown 204.

The body weight scale 210 may include a plurality of electrodes 211 disposed on an upper surface of the body weight scale 210, and a display 212. The number of the electrodes 211 may be determined according to a method of measuring bioelectrical impedance of the user by the body weight scale 210. In the present exemplary embodiment, the body weight scale 210 may measure the bioelectrical impedance according to a 4-point measurement method. Thus, the body weight scale 210 may have four electrodes 211 distributed in the upper surface thereof. The electrodes 211 may be in contact with the left and right soles of the feet of the user.

The biometric information measurement device 200 or 210 may include a storage unit for storing the biometric information, and may store the biometric information in a security area of the storage unit. The biometric information measurement device 200 or 210 may include an authentication means providing an authentication procedure for granting access to the biometric information stored in the security area. The authentication means may provide the authentication procedure based on an authentication method such as password input, pattern input, EMG signal detection, fingerprint recognition, iris recognition, ECG signal detection, or facial recognition.

In addition, the biometric information measurement device 200 or 210 may be connected to another electronic device to communicate therewith, thus sharing the biometric information with the other electronic device. In the present exemplary embodiment, an electronic device sharing the biometric information with the biometric information measurement device 200 or 210 may be a smartphone 220. However, the biometric information measurement device 200 or 210 may share the biometric information with various electronic devices other than the smartphone 220.

In an exemplary embodiment of the present inventive concept, the biometric information measurement device 200 or 210 may permit the user to share the biometric information only when the user passes the authentication procedure provided by the authentication means. When the smartwatch 200 obtains and stores the biometric information, the user may share the biometric information, stored in the smartwatch 200, with the smartphone 220 through the authentication procedure provided by the smartwatch 200. The smartphone 220 may include the storage unit for storing the biometric information received from the smartwatch 200 and, in an exemplary embodiment of the present inventive concept, may store the biometric information, received from the smartwatch 200, in a security area of the storage unit. For example, the smartphone 220 may also store the biometric information in the security area of the storage unit, as in the smartwatch 200 or the body weight scale 210, to thus safely manage the biometric information.

The user may access the biometric information, stored in the smartphone 220, through a predetermined authentication procedure provided by the smartphone 220. For example, when the user executes an application using the biometric information stored in the smartphone 220 or directs the smartphone 220 to display the biometric information on a display thereof, the smartphone 220 may request that the user first pass the predetermined authentication procedure. The authentication procedure, provided by the smartphone 220, may employ an authentication method such as password input, pattern input, fingerprint recognition, iris recognition, facial recognition, ECG signal detection, or EMG signal detection.

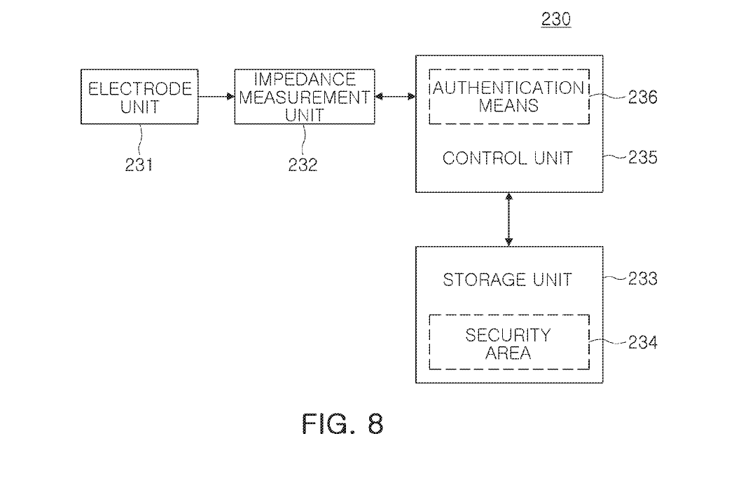

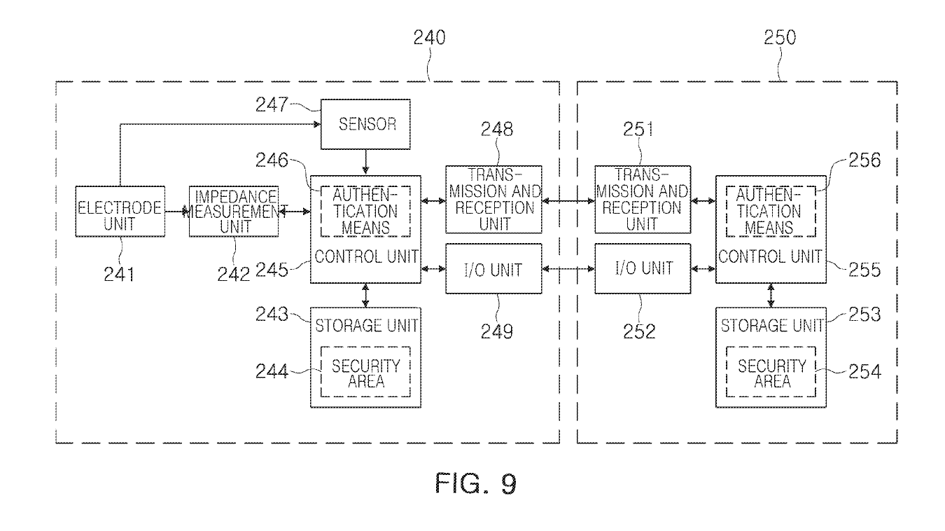

FIGS. 8 and 9 are schematic block diagrams illustrating devices for measuring biometric information according to exemplary embodiments of the present inventive concept.

Referring to FIG. 8, a biometric information measurement device 230 according to an exemplary embodiment of the present inventive concept may include an electrode unit 231, an impedance measurement unit 232, a storage unit 233, and a control unit 235. The storage unit 233 may include a separately managed security area 234, and the control unit 235 may include an authentication means 236. In an exemplary embodiment of the present inventive concept, the authentication means 236 may provide an authentication procedure based on a predetermined authentication method, and the user may be allowed to access information stored in the security area 234 only when the user passes the authentication procedure.

The electrode unit 231 may include a plurality of electrodes, and at least a portion of the plurality of electrodes may be in contact with a user's body. The impedance measurement unit 232 may detect an electrical signal such as a level of current or voltage through the electrode unit 231, and may measure bioelectrical impedance of the user based on the detected electrical signal. The control unit 235 may obtain various kinds of biometric information using the user's body information and the bioelectrical impedance measured by the impedance measurement unit 232. The body information may be directly entered by the user and may include information about the user's age, race, sex, height, or weight. The biometric information, obtained from the bioelectrical impedance and the body information by the control unit 235, may include information about body water content, muscle mass, body fat mass, BMI, skin dryness, stress index, or heart rate.

The bioelectrical impedance, the body information, and the biometric information may be stored in the security area 234. Thus, when the user of the biometric information measurement device 230 desires to confirm the biometric information or the like, the user may access the biometric information stored in the security area 234 only when first passing the authentication procedure provided by the authentication means 236. In an exemplary embodiment of the present inventive concept, the biometric information measurement device 230 may request that the user pass a predetermined authentication procedure when the user executes an application using the biometric information, the bioelectrical impedance, or the body information.

Referring to FIG. 9, a biometric information measurement device 240 may be communicatively connected to another electronic device 250. The biometric information measurement device 240 may include a sensor 247, a transmission and reception unit 248, and an input/output (I/O) unit 249 in addition to an electrode unit 241, an impedance measurement unit 242, a storage unit 243, and a control unit 245. Configurations and operations of the electrode unit 241, the impedance measurement unit 242, the storage unit 243, and the control unit 245 may be similar to those described above with reference to FIG. 8.

The sensor 247 may include various types of sensors. In an exemplary embodiment of the present inventive concept, the sensor 247 may include an acceleration sensor, a global positioning system (GPS) sensor, an ECG sensor, or an EMG sensor. At least a portion of information, collected by the sensor 247, may be used in the authentication procedure provided by an authentication means 246 included in the control unit 245. In an exemplary embodiment of the present inventive concept, an authentication procedure, using an ECG signal detected by the ECG sensor or an EMG signal detected by the EMG sensor, may be provided by the authentication means 246. Alternatively, an authentication procedure, using a motion of the biometric information measurement device 240 in a certain direction detected by the acceleration sensor, may be provided by the authentication means 246.

The biometric information measurement device 240 may be communicatively connected to the other electronic device 250 through the transmission and reception unit 248, thus sharing the biometric information with the other electronic device 250. The other electronic device 250, connected to the biometric information measurement device 240, may be a mobile device such as a smartphone, a tablet personal computer (PC), or a laptop PC; a home appliance such as a refrigerator, a television, a washing machine, an air cleaner, or a humidifier; or an exercise machine such as a rowing machine, a running machine, or a cycling machine.

The other electronic device 250 may include a transmission and reception unit 251, an I/O unit 252, a storage unit 253, and a control unit 255. The transmission and reception unit 251 may allow the biometric information, received from the biometric information measurement device 240, to be stored in a security area 254 of the storage unit 253. The control unit 255 may include an authentication means 256 providing a predetermined authentication procedure, and may permit the user to have access to the biometric information, stored in the security area 254, only when the user passes the authentication procedure provided by the authentication means 256.

In the present exemplary embodiment, the biometric information measurement device 240 may include a security area 244 and the authentication means 246, and the other electronic device 250 may include the security area 254 and the authentication means 256. The biometric information, shared by the biometric information measurement device 240 and the other electronic device 250, may be stored in the security areas 244 and 254, and the user may be allowed to access the security areas 244 and 254 only when the user passes the authentication procedures provided by the authentication means 246 and 256. Thus, the other electronic device 250 receiving and sharing the biometric information, as well as the biometric information measurement device 240 directly obtaining the biometric information, may safely manage the biometric information, thus strengthening security.

In an exemplary embodiment of the present inventive concept, the authentication means 246 and 256 may provide a plurality of authentication procedures using different authentication methods, and the provided authentication procedures may be executed simultaneously or sequentially. The authentication procedures, provided by the authentication means 246 and 256, may include at least one authentication method such as password input, pattern input, iris recognition, fingerprint recognition, facial recognition, ECG signal detection, or EMG signal detection.

The authentication method of detecting an ECG signal may have a higher false acceptance rate (FAR), but may be very difficult to duplicate, as compared to the other authentication methods. Thus, an exemplary embodiment of the present inventive concept may combine the authentication method using ECG signal detection and an authentication method having a low FAR to provide an authentication procedure to the user.

According to exemplary embodiments of the present inventive concept, units and other components described above with reference to FIGS. 8 and 9 may be embodied as hardware, e.g., circuits.

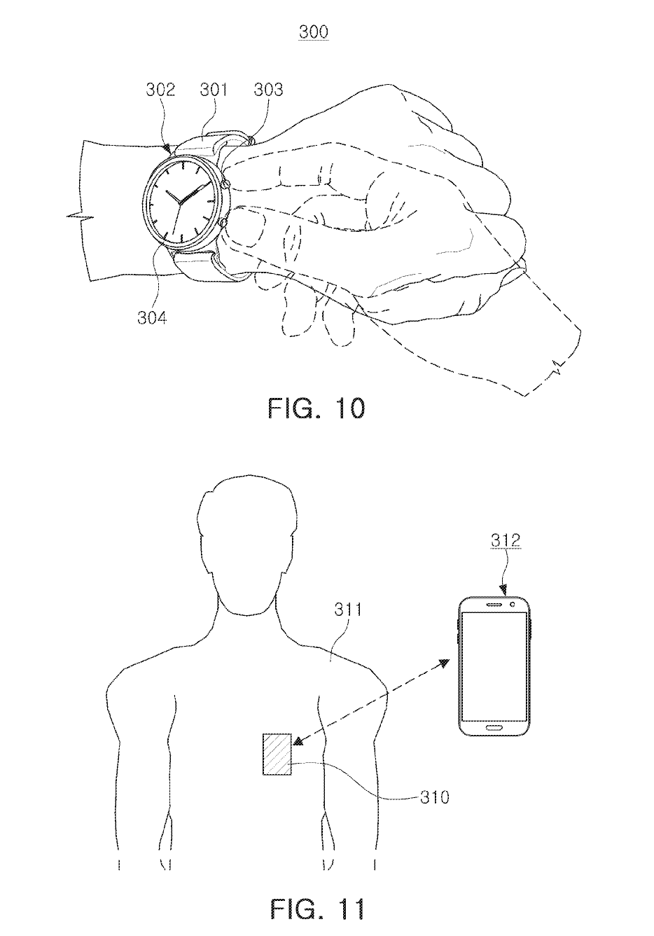

FIGS. 10 and 11 are diagrams illustrating operations of devices for measuring biometric information according to exemplary embodiments of the present inventive concept.

Referring to FIG. 10, a biometric information measurement device 300 according to an exemplary embodiment of the present inventive concept may be a wearable device that may be worn on a user's body, and may be a smartwatch. The biometric information measurement device 300 may include a strap 301 fixing the biometric information measurement device 300 to the user's body, and a body portion 302. When the biometric information measurement device 300 is a different type of wearable device, other than a smartwatch, a configuration of the biometric information measurement device 300 may be different.

The biometric information measurement device 300 may include a plurality of electrodes in contact with the user's body, and may measure a bioelectrical impedance of the user using an electrical signal detected through the plurality of electrodes. In an exemplary embodiment of the present inventive concept, when the biometric information measurement device 300 is the smartwatch, a first portion of the plurality of electrodes may be disposed on an internal surface of the strap 301 or an internal surface of the body portion 302 in contact with the user's body. Further, the first portion of the plurality of electrodes may be externally exposed when the user wears the biometric information measurement device 300.

In the present exemplary embodiment, the first portion of the plurality of electrodes may be disposed in a crown 303 of the body portion 302. According to an exemplary embodiment of the present inventive concept, a second portion of the plurality of electrodes may also be disposed on an upper surface of the body portion 302, a dial 304, or an external surface of the strap 301. The user may execute an application for obtaining biometric information while wearing the biometric information measurement device 300, and then may touch the crown 303 or the dial 304 having at least some of the plurality of electrodes disposed therein. When the user's body comes into contact with the at least some of the plurality of electrodes of the biometric information measurement device 300, the biometric information measurement device 300 may detect a level of current or voltage through these electrodes to measure a bioelectrical impedance.

The biometric information measurement device 300 may produce the biometric information using the bioelectrical impedance and the user's body information. The biometric information may include information about the user's age, height, weight, race, or sex. The biometric information measurement device 300 may produce the user's biometric information such as body water content, muscle mass, or body fat mass, using the bioelectrical impedance and the body information. The biometric information measurement device 300 may store the biometric information in a storage unit and, in an exemplary embodiment of the present inventive concept, may store the biometric information in a separate security area of the storage unit.

The biometric information measurement device 300 may grant access to the biometric information, stored in the security area, to the user, only when the user passes a predetermined authentication procedure. For this purpose, the biometric information measurement device 300 may include an authentication means providing a predetermined authentication procedure. In an exemplary embodiment of the present inventive concept, the authentication means may provide a plurality of authentication procedures simultaneously or sequentially, and the authentication procedures may use different authentication methods.

In an exemplary embodiment of the present inventive concept, at least one of the authentication procedures provided by the authentication means may use an ECG signal of the user. For example, the biometric information measurement device 300 may compare the ECG signal, detected from the user, with a previously stored reference ECG signal to determine whether the user is a previously registered authenticated user. The ECG signal may be measured with the user's body being in contact with the electrodes of the biometric information measurement device 300, similar to the example described with reference to FIG. 7.

Referring to FIG. 10, the user may bring his or her finger into contact with the crown 303 having the electrodes disposed therein, to detect the ECG signal. Here, two fingers of the user may come into contact with two crowns 303, spaced apart from each other, and in this process, levels of resistance of the fingers may offset each other.

The authentication method, using the ECG signal, may be a physical authentication method that has been widely used in the related art, such as password input or pattern input, but may be difficult to duplicate, as compared to other biometric authentication methods such as iris recognition or fingerprint recognition, thus achieving a high level of security. However, the authentication method using the ECG signal may have a relatively low level of sensitivity. Thus, exemplary embodiments of the present inventive concept may also use another authentication method having a relatively high level of sensitivity, to compensate for the relatively low level of sensitivity of the authentication method using the ECG signal.

In an exemplary embodiment of the present inventive concept, a pattern input method using an order or frequency in which the user presses or touches the crown 303 of the biometric information measurement device 300, a pattern input method using a direction of rotation in which the user touches the dial 304 of the biometric information measurement device 300, or a password input method using a keypad displayed on a display of the biometric information measurement device 300, may be used with the authentication method using the ECG signal.

Thus, the biometric information measurement device 300 may provide a security procedure having an excellent level of security, while compensating for the relatively low level of sensitivity of the authentication method using the ECG signal.

Referring to FIG. 11, an ECG signal may be detected by a patch 310 attachable to a body part of a user 311. The patch 310 may be connected to an electronic device 312, such as a smartphone, to communicate with the electronic device 312, and, in an exemplary embodiment of the present inventive concept, may communicate with the electronic device 312 in a body area network (BAN) environment. The patch 310 may include a communications module for transmitting and receiving a signal and a battery module for supplying power required for operations of the patch 310, in addition to an ECG sensor for detecting an ECG signal. In an exemplary embodiment of the present inventive concept, the patch 310 may be attached adjacent to the user's heart.

An authentication method, using the ECG signal detected by the patch 310, may be used in an authentication procedure for granting permission to use the electronic device 312 or access to data stored in the electronic device 312. In an exemplary embodiment of the present inventive concept, the patch 310 may further include another biosensor for measuring bioelectrical impedance of the user 311 in addition to the ECG sensor. The electronic device 312 may store various types of biometric information of the user 311 that may be produced from the ECG signal or the bioelectrical impedance measured by the patch 310. In an exemplary embodiment of the present inventive concept, the biometric information of the user 311 may be stored in a security area of a storage unit included in the electronic device 312. The authentication method, using the ECG signal detected by the patch 310, may be provided as a portion of the authentication procedure of accessing the biometric information of the user 311 stored in the security area.

In an exemplary embodiment of the present inventive concept, the authentication method using the ECG signal may be applied to various applications. In an exemplary embodiment of the present inventive concept, the authentication method using the ECG signal may be used in a process of providing an electronic financial service by an electronic financial service device. Alternatively, the authentication method using the ECG signal may be used in a process of allowing devices, included in an IoT system, to share information with one another, or registering, deleting, or changing devices over an IoT network. Additionally, the authentication method using the ECG signal may be used with another authentication method having an excellent level of sensitivity, to compensate for the relatively low level of sensitivity of the authentication method using the ECG signal.

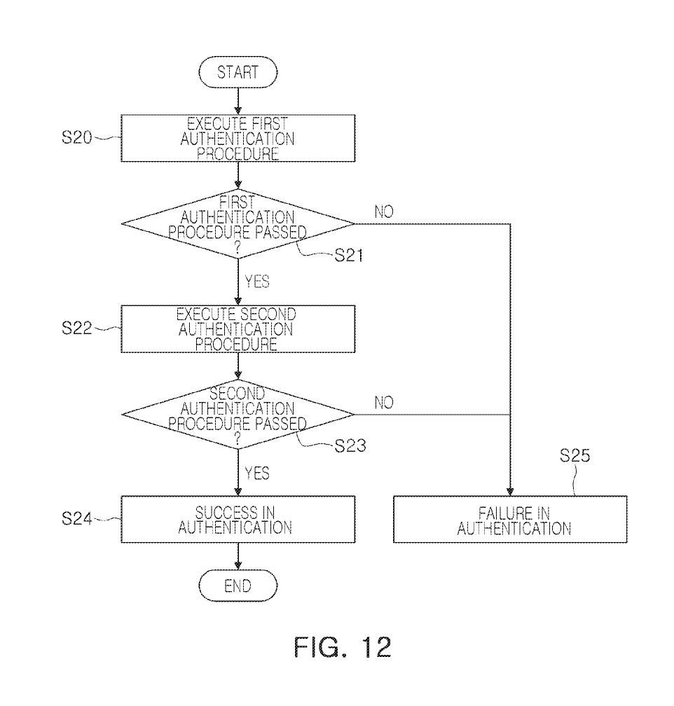

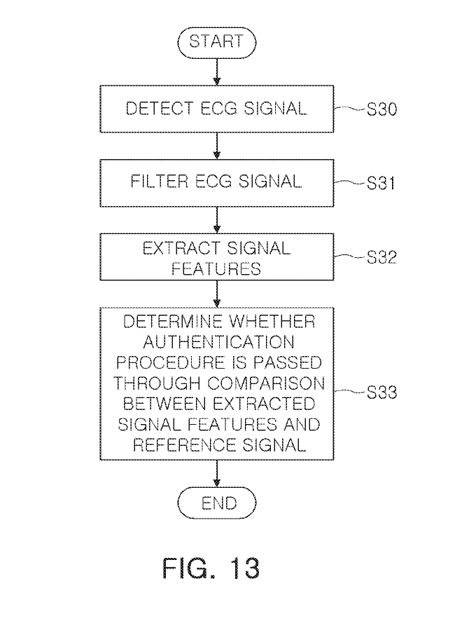

FIGS. 12 and 13 are flowcharts of user authentication methods of a device for measuring biometric information according to exemplary embodiments of the present inventive concept. The user authentication methods according to exemplary embodiments of the present inventive concept may be performed by a biometric information measurement device or an electronic device receiving biometric information from the biometric information measurement device and storing the received biometric information.

Referring to FIG. 12, the user authentication method according to an exemplary embodiment of the present inventive concept may start by executing a first authentication procedure (S20). The first authentication procedure may be executed by detecting an ECG signal of a user and comparing the detected ECG signal with a previously stored reference signal. After executing the first authentication procedure, it may be determined whether the user has passed the first authentication procedure (S21), and when it is determined that the user has passed the first authentication procedure, a second authentication procedure may be executed (S22).

The second authentication procedure may use an authentication method different from that of using the ECG signal. In an exemplary embodiment of the present inventive concept, the second authentication procedure may be executed using an authentication method such as password input, pattern input, fingerprint recognition, iris recognition, or EMG signal detection. The user authentication method according to an exemplary embodiment of the present inventive concept may determine whether the user that has passed the first authentication procedure also passes the second authentication procedure (S23), and, when it is determined that the user has passed the second authentication procedure, may determine that the user succeeds in authentication (S24). The user authentication method according to an exemplary embodiment of the present inventive concept may determine that, when the user fails the first authentication procedure or the second authentication procedure, the user has failed authentication (S25). Thus, the user authentication method may have increased security compared to the case of only using a single authentication procedure.

The user authentication method, described with reference to FIG. 12, may be modified in various manners. In an exemplary embodiment of the present inventive concept, the first authentication procedure using the authentication method based on the ECG signal may be executed simultaneously with the second authentication procedure using a different authentication method. For example, when the user brings his or her finger into contact with electrodes of the device, the user authentication method may detect an electrical signal from the user's finger to obtain a fingerprint image from the finger, while measuring the ECG signal. Here, the first authentication procedure using the fingerprint recognition and the second authentication procedure using the ECG signal may be simultaneously executed.

Alternatively, the second authentication procedure using a different authentication method not based on the ECG signal may first be executed, and then the first authentication procedure using the authentication method based on the ECG signal may be executed.

FIG. 13 is a schematic flowchart illustrating a user authentication method using an ECG signal. Referring to FIG. 13, the user authentication method according to an exemplary embodiment of the present inventive concept may start by detecting the ECG signal from the user (S30). Detection of the ECG signal may be performed by an ECG sensor, which may include electrodes in contact with the user's body, an analog front end (AFE), or a digital filter.

In an exemplary embodiment of the present inventive concept, the operation S30 may be performed by the AFE, which may detect the ECG signal from the user through the electrodes and convert the detected ECG signal to a digital format.

When the ECG signal is detected, the ECG sensor may filter the detected ECG signal (S31). In an exemplary embodiment of the present inventive concept, the operation S31 may be performed by the digital filter included in the ECG sensor. The digital filter may pass the ECG signal in the digital format, generated by the AFE, only in an ECG frequency band, and may remove or filter out the ECG signal in frequency bands outside the ECG frequency band to increase a signal-to-noise ratio (SNR).

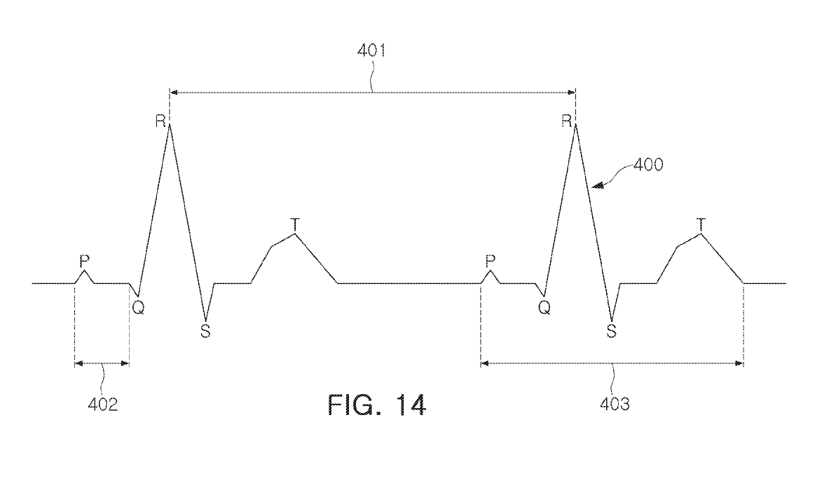

When the filtering is completed, the user authentication method may extract features from the ECG signal in the digital format (S32). The features, extracted in the operation S32, may be compared with those of a previously stored reference ECG signal and, according to a similarity between the compared features, the user authentication method may determine whether the user passes an authentication procedure (S33). In an exemplary embodiment of the present inventive concept, the features of the ECG signal extracted in the operation S32 may include information about the ECG signal's PR interval, PR segment, QRS complex, ST segment, ST interval, QT interval, or RR interval. Hereinafter, a description will be provided with reference to FIG. 14.

FIG. 14 is a graph of an ECG signal used in the user authentication method of FIG. 13 according to an exemplary embodiment of the present inventive concept. Referring to FIG. 14, an ECG signal 400 may have repeated P waves, QRS waves, and T waves. The user authentication method according to an exemplary embodiment of the present inventive concept may extract an RR interval 401, a PQ interval 402, and a PT interval 403 from the ECG signal 400 as features thereof, compare the extracted features with those of a previously stored reference ECG signal, and determine the similarity between the compared features, thus performing user authentication.

As set forth above, according to exemplary embodiments of the present inventive concept, a device for measuring biometric information may store obtained biometric information in a security area of a storage unit, and may permit the user to have access to the stored biometric information only when the user passes an authentication procedure provided by an authentication means included in the device for measuring biometric information, so as to share the biometric information with other devices. Thus, the device for measuring biometric information may provide various applications to the user while safely managing biometric information measured from the user.

While the present inventive concept has been shown and described above with reference to exemplary embodiments thereof, it will be apparent to those of ordinary skill in the art that modifications and variations could be made thereto without departing from the scope of the present inventive concept, as set forth by the following claims.

What is claimed is:

1. A device for measuring biometric information, the device comprising:
an impedance measurement circuit configured to receive an electrical signal from a plurality of electrodes when the plurality of electrodes are in contact with a body of a user and to measure a bioelectrical impedance using the electrical signal;
a storage circuit including a security area configured to store the bioelectrical impedance, body information of the user, and biometric information of the user obtained from the bioelectrical impedance and the body information; and
a control circuit including an authentication means configured to determine access authority to data stored in the security area using an authentication procedure, and configured to obtain the biometric information from the bioelectrical impedance and the body information and to manage the security area through the authentication procedure provided by the authentication means.

2. The device of claim 1, wherein the impedance measurement circuit measures the bioelectrical impedance using at least one of a 4-point measurement method or a 2-point measurement method.

3. The device of claim 1, further comprising:
a transmission and reception circuit configured to relay communications with an external electronic device,
wherein the control circuit transmits the data, stored in the security area, to the external electronic device via the transmission and reception circuit through the authentication procedure.

4. The device of claim 1, wherein the control circuit compares the biometric information with a target biometric information previously set by the user.

5. The device of claim 1, wherein the authentication procedure includes a first authentication procedure and a second authentication procedure, and
the first authentication procedure and the second authentication procedure authenticate the user using different information.

6. The device of claim 5, wherein the authentication means authenticates the user, in the first authentication procedure, using an electrocardiogram (ECG) signal of the user, and authenticates the user, in the second authentication procedure, using information having a false acceptance rate (FAR) that is lower than that of the ECG signal.

7. The device of claim 6, wherein the authentication means authenticates the user using at least one of password input, pattern input, electromyography (EMG) signal detection, fingerprint recognition, or iris recognition in the second authentication procedure.

8. The device of claim 6, wherein the authentication means executes the first authentication procedure and the second authentication procedure at substantially the same time.

9. The device of claim 6, wherein the authentication means authenticates the user using the first authentication procedure after the user has passed the second authentication procedure.

10. A device for measuring biometric information, the device comprising:
an electrode circuit including a plurality of electrodes configured to be in contact with a body of a user;
an electrocardiogram (ECG) sensor configured to measure an ECG signal using an electrical signal detected by the electrode circuit; and
a control circuit including an authentication means,
wherein the authentication means is configured to provide a first authentication procedure, and a second authentication procedure, and to authenticate the user that has passed the first authentication procedure and the second authentication procedure as an authenticated user,
the first authentication procedure authenticates the user with a first authentication method using the ECG signal, and
the second authentication procedure authenticates the user with a second authentication method that does not use the ECG signal.

11. The device of claim 10, wherein the second authentication method has a false acceptance rate (FAR) lower than that of the first authentication method.

12. An Internet of Things (IoT) system comprising:
a plurality of electronic devices configured to communicate with one another through an IoT network; and
at least one biometric information measurement device configured to communicate with at least a portion of the plurality of electronic devices through the IoT network, and to obtain biometric information of a user through contact with a body of the user,
wherein the at least one biometric information measurement device includes an authentication means configured to confirm whether the user is authenticated,
the at least one biometric information measurement device shares the biometric information with the at least a portion of the plurality of electronic devices through an authentication procedure provided by the authentication means, and
the plurality of electronic devices provide services to the user using the biometric information.

13. The IoT system of claim 12, wherein the plurality of electronic devices include at least one exercise machine, and the at least one exercise machine guides the user through a type of exercise and an amount of exercise, using the biometric information.

14. The IoT system of claim 12, wherein the plurality of electronic devices include at least one home appliance, and the at least one home appliance controls an environment of the user's living space, using the biometric information.

15. The IoT system of claim 12, wherein the at least one biometric information measurement device collects bioelectrical impedance and body information from the user to obtain the biometric information.

16. The IoT system of claim 12, wherein the biometric information includes at least one of the user's muscle mass, body fat mass, body water content, heart rate, stress information, skin temperature, skin dryness, electrocardiogram (ECG) signal, or electromyography (EMG) signal.

17. The IoT system of claim 12, wherein each of the plurality of electronic devices and the at least one biometric information measurement device includes a storage circuit configured to store the biometric information, and stores the biometric information in a security area of the storage circuit.

18. The IoT system of claim 12, wherein the authentication means provides a first authentication procedure and a second authentication procedure, and
the first authentication procedure and the second authentication procedure confirm whether the user is authenticated using different authentication methods.

19. The IoT system of claim 18, wherein the authentication means confirms whether the user is authenticated by detecting an ECG signal of the user in the first authentication procedure, and confirms whether the user is authenticated using at least one of password input, pattern input, EMG signal detection, fingerprint recognition, or iris recognition in the second authentication procedure.

20. The IoT system of claim 18, wherein the authentication means confirms that the user is authenticated when the user has passed both the first authentication procedure and the second authentication procedure.
